(19) United States
(12) Patent Application Publication
(10) Pub. No.: US 2011/0313034 A1
(43) Pub. Date: Dec. 22, 2011

(54) NEUROACTIVE PLANT EXTRACT FROM HYPERICUM POLYANTHEMUM

(75) Inventors: Stela Maris Kuze Rates, Porto Alegre, RS (BR); Gilsane Lino Von Poser, Porto Alegre, RS (BR); Alice Fialho Viana, Porto Alegre, RS (BR); Jean Costentin, Rouen (FR); Jean-Claude Do Rego, Malaunay (FR)

(73) Assignees: UNIVERSIDADE FEDERAL DO RIO GRANDE DO SUL, Porto Alegre, RS (BR); UNIVERSITE DE ROUEN, Mont-Saint-Aignan (FR)

(21) Appl. No.: 13/148,750

(22) PCT Filed: Feb. 12, 2010
(86) PCT No.: PCT/EP2010/051816

§ 371 Date: Aug. 10, 2011

(30) Foreign Application Priority Data
Feb. 13, 2009 (BR) PI0900614-1

(51) Int. Cl. A61K 31/352; A61P 25/00

(52) U.S. Cl. 514/456

(57) ABSTRACT

The present invention belongs to the field of plant extracts with activity on the central nervous system. Specifically, the plant extract of the present invention is an extract obtained from Hypericum polyanthemum and comprising the compound uliginosin B and/or compounds from the class of benzopyrans such as. The present invention also relates to a pharmaceutical composition comprising such an extract which has pharmacological action in psychiatric disorders, such as mood disorders, specifically, anti-depressant activity. Such as HP1 (6-isobutyryl-5,7-dimethoxy-2,2-dimethyl-benzopyran), HP2 (7-hydroxy-6-isobutyryl-5-methoxy-2,2-dimethyl-benzopyran) and HP3 (5-hydroxy-6-isobutyryl-7-methoxy-2,2-dimethyl-benzopyran)).

### FIELD OF THE INVENTION

The present invention belongs to the field of plant extracts with activity on the central nervous system. Specifically, the plant extract of the present invention is an extract obtained from Hypericum polyanthemum and comprising the compound uliginosina B or derivatives thereof. The present invention also relates to a pharmaceutical composition containing said extract which has activity on mood disorders, specifically, antidepressant activity; as well as the process to obtain the extract.

### BACKGROUND OF THE INVENTION

Psychoactive drugs are a class in critical need of development. They were introduced in therapy by the end of 50's and generated a revolution in the treatment of psychiatric diseases and a change in the way these diseases were considered, since they provided the possibility of studying their biological basis (Feighner, 1999). For instance, the understanding of the neurochemical basis of depression and schizophrenia is closely linked to the understanding of the mechanism of action of antidepressants and antipsychotic drugs. However, all of the biochemical phenomena related to these diseases is far from completely understood. Likewise, the exact mechanism of action of many of these drugs is still not fully understood. In addition, about 35% of psychiatric patients do not respond adequately to drug treatment, and in most cases, even the adequate therapeutic responses come along with important side effects (Berton and Nestler, 2006).

However, the majority of research is still based on the original approach of the monoamine theory for the genesis of psychiatric disorders, based on the observation of the effects of reserpine, monoamine oxidase inhibitors and tricyclic antidepressants. Maintaining this approach will not result in truly innovative drugs, nor will it improve knowledge about mental illness (Nestler and Carlezon, 2006). This fact becomes troublesome considering that mood disorders such as depression have prevalence rates of approximately 18%, and are considered one of the most disabling and costly mental illnesses (Gold and Charney, 2002).

It is in this context that the study of the genus Hypericum is to be appreciated. The specie Hypericum perforatum, commonly known in the U.S. and England as St. John's wort and in Germany as Johanniskraut (herb of St. John), is an alternative to synthetic antidepressants for treating mild to moderate depression. Clinical studies demonstrate the efficacy of standardized extracts of H. perforatum in these situations (Linde et al., 1996; Kasper et al., 2006) and studies on its mechanism of action indicate that these extracts act differently from current antidepressants (Chatterjee et al., 1998; Kumar et al., 2001). An interesting feature of their activity is the non-specific action. The extract and some isolated compounds inhibit synaptosomal reuptake of serotonin, norepinephrine, dopamine, GABA and glutamate (Wonnemann et al., 2000; Roz and Rehavi, 2003). The binding of histamine, neurokinin, corticotropin and opiates to their receptors is also inhibited by extracts of H. perforatum (Simmen et al., 2001).

Epidemiological Data on Depression

Depressive disorders can affect people of any gender, race and socioeconomic level. The WHO estimates that currently 121 million people suffer from depression. Approximately 5.8% of men and 9.5% of women will suffer a depressive episode at some point in life, although these values may vary among different populations. Chronic or recurrent depression may result in damage on both personal and professional levels. It is the main cause of absence from work (measured in YLD, Years Lived with Disability) and the fourth cause when considering the potential years of productive life lost due to premature death or disease (DALY, Disability Adjusted Life Years) and WHO projects that by 2020 depression will reach the second place as the cause of DALYs. Moreover, suicide remains a possible consequence of Major Depressive Disorder. Together, Major Depressive Disorder and Schizophrenia are responsible for 60% of suicides worldwide.

Depression Treatments

The first line of treatment includes antidepressant medication, psychotherapy or a combination of both. Other effective interventions include setting up a support network for vulnerable individuals, families or groups. The evidence on the prevention of depressive episodes is less conclusive. Treatment with antidepressants along with psychotherapy is effective in 60-80% of patients. However, less than 25% of affected individuals (in some countries less than 10%) receive treatment, partly due to lack of resources, of trained personnel and to the stigma associated with mental illnesses that lead patients to not seek help.

In the 50's two classes of antidepressants were discovered, the tricyclics and monoamine oxidase inhibitors (MAOIs). Although currently there are more than 25 substances used to manufacture antidepressant drugs, since the discovery of MAOI and TCA there has been no breakthrough in the mechanism of action of these drugs, since they all still act via monoaminergic transmission, only with greater specificity than tricyclics and MAOIs. The great advantage of second-generation and atypical antidepressants is milder side effects and therefore better acceptance by patients. The following is a summary of the main characteristics of the classes of available antidepressants:

Tricyclic Antidepressants (TCA)

In the late 40's, Haflinger and Schindler synthesized a series of more than 40 iminodibenzil derivatives for possible use as an antihistaminergic, analgesic, anti-parkinsonian and/or sedative. Among the substances selected in preclinical trials for their sedative or hypnotic properties was imipramine. Unexpectedly, it provided an indisputable improvement in some depressed patients (Baldessarini, 1996). Later, it was discovered that all clinically active tricyclic antidepressants inhibit the reuptake of serotonin and noradrenaline with different effectiveness (Nestler, 1998). However, due to their origin, they also act on histamine and acetylcholine receptors. The latter interaction has important atropine-like effects, including dry mouth and constipation, dizziness, blurred vision, sedation, orthostatic hypotension. Other adverse reactions include cardiovascular changes, and over-dose of TCAs decreases intraventricular conduction and may cause heart failure or ventricular arrhythmias. In over-dose these medications can also cause seizures especially in patients who have had previous episodes. Some examples are: imipramine, amitriptyline, clorimipramine, desimipramine.

Monoamine Oxidase Inhibitors (MAOIs)

They were the first clinically active antidepressants and had a great impact on the development of modern biological psychiatry (Baldessarini, 1996). In 1951 isoniazid and its isopropyl derivative, iproniazid, were developed for the treatment of tuberculosis. It was observed that iproniazid improved the mood in TB patients with symptoms of depression. In 1952, the group of Zeller found that iproniazid had an inhibitory activity on the enzyme monoamine oxidase (Baldessarini, 1996). This class of antidepressants increases the levels of catecholamines by inhibiting MAO, an enzyme that degrades brain amines.

The first drugs with MAOI activity (phenelzine, tranilciclopride) are irreversible inhibitors of the enzyme, i.e. its activity returns only after the synthesis of a new enzyme. For this reason, the intake of foods containing tyramine, particularly those produced by fermentation (eg cheese, wine, beer) should be avoided, since the MAO also oxidizes other phenethylamines. This interaction is less critical for selective and reversible MAO-A inhibitors, such as moclobemide. Other adverse reactions include: hypotension, weight gain and sexual dysfunction. Nevertheless, some patients with depression respond better to MAOIs than to any other class of antidepressants (Feighner, 1999).

Selective Serotonin Reuptake Inhibitors (SSRIs)

These antidepressants increase the bioavailability of 5-HT in the synaptic cleft, because they inhibit the serotonin transporter (Stahl, 1998). This effect causes the sudden increase of serotonin predominantly in the somatodendritic area. Chronic treatment with SSRIs, with the persistent increase of serotonin in the somatodendritic area of neurons, leads to desensitization of somatodendritic auto-receptors type 5-HT1A. Since these auto-receptors remain desensitized, neural impulse flow is no longer inhibited by the presence of 5-HT. In other words, serotonergic neurotransmission is uninhibited, and more serotonin is released from the axon terminal (Stahl, 1998). Its efficacy, especially in major depression, it is not superior to TCA, but the risk of overdose is lower. In addition, most patients seem to tolerate adverse reactions, which are often transient, such as nausea, dizziness, diarrhea, agitation or sedation. The use of SSRIs may cause sexual dysfunction in men and women, including reduced libido, anorgasmia, delayed ejaculation, impotence (Papakostas and Fava, 2007). Ex: Fluoxetine, paroxetine, sertraline.

Atypical Antidepressants

Selective serotonin and norepinephrine reuptake inhibitors (SNRIs)—The reuptake of serotonin and noradrenaline are a recent class of antidepressants, whose first representative was the velafaxine, but it already is a first-line treatment for depression. They increase the activity of both NA and 5-HT, and interact weakly with dopamine receptors and appear to act as noncompetitive antagonist of nicotinic receptors (Papakostas and Fava, 2007). There seems to be a dose-response relationship with this drug, and in high doses, the adrenergic effect is increased (Feighner, 1999). Velafaxine causes an acute down-regulation β-adrenergic receptors, which suggests a possible early onset of its effects, but no conclusive studies are available. Depending on the dose used, its adrenergic effects may increase blood pressure, however this effect is not observed in all studies. In the U.S. the FDA (Food and Drug Administration) advises that is necessary to monitor blood pressure (Feighner, 1999). Some of the side effects of these antidepressants, as well as the SSRIs, are due to non-selective activation of multiple 5-HT and NA receptors (Yadid et al., 2000). Other representatives are duloxetine and milnacipram (Papakostas and Fava, 2007).

Serotonin reuptake inhibitors and 5-HT₂ blockers—Two important representatives of this group are trazodone and its analogue, nefazodone. They act as relatively weak inhibitors of serotonin and noradrenaline (Papakostas and Fava, 2007) and block the post-synaptic receptors 5-HT_{2A}/5-HT_{2C} (Feighner, 1999; Millan, 2006). By blocking 5-HT_{2A} and inhibiting serotonin reuptake, nefazodone appears to have a dual mechanism of action on the serotoninergic system. Trazodone is also a strong blocker of the adrenergic receptor α₁, which is thought to be responsible for the sedative effect of trazodone and can also be related to priapism, this effect being less pronounced with the use of nefazodone (Papakostas and Fava, 2007). Other adverse reactions include difficulty concentrating, and lethargy. It also does not have quinidine-like action, and is safe in over-dose, and has a low rate of epilepsy and sexual dysfunction, especially when compared with SSRIs, venlafaxine, TCA and MAOI. Nefazodone has no antihistamine or anti-cholinergic activity, which improves its tolerability and safety (Feighner, 1999).

Adrenergic and serotoninergic-specific antidepressant (NaSSa)—An example of antidepressant that acts on specific NA and 5-HT receptors is mirtazepine. It blocks the α₂-adrenergic auto-receptors and α₂-hetero-serotoninergic receptors responsible for regulating the release of NA and 5-HT. Furthermore, it blocks the postsynaptic receptors, 5-HT_{2A}, 5-HT_{2C} and 5-HT₃. This blockage results in increased noradrenergic and serotoninergic specific activity, which results in fewer SSRI-like side effects (gastrointestinal disorders, insomnia and sexual dysfunction) and TCA-like side effects (dry mouth, constipation and dizziness). However, the mirtazepine has histaminergic action, which can cause sedation and increased appetite with weight gain (Feighner, 1999).

Norepinephrine reuptake inhibitors (NRIs)— Reboxetine is the most specific inhibitor of NA reuptake, with weak affinity for the serotonin transporter and dopamine or muscarinic receptor. Despite being classified as a tricyclic, desipramine may also be included in this class, due to its high specificity for the transported NA. The increase of norepinephrine seems to be related to activation of postsynaptic α₁- and α₂-adrenergic receptors in the cortico-limbic region. Some studies suggest that reboxetine may be effective in the treatment of cognitive alterations and psychosocial function during depression. Adverse reactions include headache, insomnia, dry mouth, urinary hesitancy and constipation. It is not associated with adverse effects typical of SSRIs, such as sexual dysfunction (Yadid et al., 2000).

Norepinephrine and dopamine reuptake inhibitors (IRND)—Its main representative is bupropion. A big difference compared to SSRI and NRI is the increase of DA in the nucleus accumbens, while blocking reuptake of dopamine (Millan, 2006). The activation of D1 receptors that facilitates the release of NA may contribute to elevation of NA in the frontal cortex (Feighner, 1999). In addition, bupropion is rapidly metabolized to derivatives that inhibit the reuptake of NA. It has no effect on serotonin, muscarinic, histamine and α₂-adrenergic receptors. However, bupropion appears to act as a noncompetitive antagonist of nicotinic receptors (Millan, 2006), this effect may be related to its use in treatment of smoking (Cordioli et al., 2005). An advantage in comparison to SSRI is it is not associated with sexual dysfunction or sedation. The most common adverse events of bupropion are agitation, insomnia, weight loss, dry mouth, constipation, headache and tremor. A significant adverse effect is seizure, especially in immediate release formulations. Although it may raise blood pressure, this effect is not very common (Papakostas and Fava, 2007).

Tianeptine—Although still classified by some authors as an enhancer of serotonin reuptake (Papakostas and Fava, 2007), this effect seems to be indirect since the affinity of tianeptine for 5-HT transporter is very low and it does not affect in a significant manner the extracellular levels of 5-HT (Millan, 2006). Although the mechanism of action of tianeptine is not elucidated, several experimental findings justify its antidepressant effect. Chronically it increases the sensitivity of receptors α₁-adrenergic receptors, but does not alter synaptic levels of NA (Rogoz et al., 2001). The dopaminergic system is also affected by chronic treatment with tianeptine. An increase in D₂ receptors functionality in the nucleus accumbens and in dopamine release in the mesolimbic area has been verified. The way in which this antidepressant increases dopaminergic transmission is not clear, because it does not bind to DA transporters nor to D₂ and D₃ auto-receptors. Other changes induced by tianeptine that may be related to its antidepressant effect are: reduction of the inhibitory influence of GABA (gamma-amino butyric acid) and glycine on the neuronal excitability; neuroprotective effect against the neurotoxic effects of stress and cytokines and modulation of glutamatergic transmission (Millan, 2006).

Patients Resistant to Treatment

A third or more of patients do not respond and more than half cannot achieve or maintain a complete remission with any pharmacological treatment alone. When assessing the resistance of a patient to treatment it is necessary to consider the five Ds: diagnosis, drug, dose, duration of treatment and different treatments. The physician should consider changing them all if the patient does not respond within a period of 6-8 weeks. In extreme cases, as in psychotic depression, catatonia, where the patient's life is at risk, non-pharmacological methods such as electroconvulsive therapy, transcranial stimulation and sleep deprivation can be used (Potter and Hollister 2006).

The Genus Hypericum

The family Guttiferae (Clusiaceae) is composed of 50 genera and approximately 1000 species distributed throughout the tropical and subtropical regions of the planet (Cronquist, 1981). Among some important genera of the family the most important are Calophyllum, Garcinia, Hypericum and Vismia. A large number of species of these genera has been used in traditional medicine for the treatment of cancer, viral, bacterial and fungal illnesses. The widespread use of these plants has led to the discovery of numerous molecules with various biological activities. Among these, there are the phenolic substances. These substances—in particular the meta-dihydroxylated—often have a prenyl substitution, considered as interesting pharmacophore groups. The prenylation is facilitated by the influence of hydroxyl groups, which increase the electron density, thus energetically favoring enzymatic reactions of electrophilic substitution with pyrophosphate dimethylalila (Zuurbier et al., 1998). Subsequently, in a biosynthetic sequence, cyclization can occur in the chain leading to the respective prenylated derivatives dimethyl-benzopyran.

This substitution pattern—hydroxylation, prenylation and subsequent cyclization of the prenyl group—is found in the various phenolics found in the species Guttiferae and Calanolide; such as, pyranocoumarins with significant anti-HIV-1 activity (Mckee et al., 1998) and xanthones, which have shown to have anti-inflammatory, anti-hepatotoxic, antiviral, antimicrobial, antioxidant, monoamine oxidase inhibitor (MAOI), antiprotozoal and antitumor activities (Rocha et al., 1994; Bennet and Lee, 1989). Other important substances are benzophenones, precursors of xanthones, which have antiprotozoal and anti-HIV-1 activity (Bennett and Lee 1989, Fuller et al., 1999), and derivatives of phloroglucinol, with antidepressant, antimicrobial, wound healing, anti-proliferative activities, among others (Rocha et al., 1994; Ishiguro et al., 1986; Jayasuriya et al., 1991; Rocha et al., 1996).

For pharmacological studies, the genus Hypericum, comprising about 400 species, has received special attention due to the antiviral activity of polycyclic quinones—hypericin and pseudo-hypericin—on several retroviruses in vitro and in vivo, in particular HIV (Awang et al., 1991) and the therapeutic use of H. perforatum, the better known species of the genus, as an antidepressant (Linde et al., 1996; Gaster and Holroyd, 2000).

Hypericum Species Native to Rio Grande do Sul

Phytochemical studies carried out in the laboratory of Pharmacognosy, Faculty of Pharmacy—UFRGS showed the presence of the following substances:

Phloroglucinol derivatives: So far, all phloroglucin derivatives identified in the native species present a dimeric structure consisting of a filicinic acid linked to a phloroglucin. From the cyclohexane extract of H. myrianthum aerial parts, were isolated phloroglucinol already described: japonicina A (Dall 'Agnol et al., 2003), present in H. japonicum (Ishiguro et al., 1987) and H. brasiliense (Rocha et al., 1995), and uliginosina B (Ferraz et al., 2002a) also present in H. carinatum and H. polyanthemum (Nor et al., 2004). Hiperbrasilol B, previously isolated from H. brasiliense (Rocha et al., 1996), was identified in H. carinatum and H. caprifoliatum. From H. caprifoliatum was also isolated a number of tautomers, the spectroscopic analysis by nuclear magnetic resonance (NMR) showed the presence of a phloroglucinol derivative linked to filicinic acid (Viana, 2002). The separation and structural elucidation of these tautomers are still in progress.

Benzopyrans: From the chloroform extract of H. polyanthemum aerial parts three benzopyrans novel structures have been isolated: HP1 (6-isobutyryl-5,7-dimethoxy-2,2-dimethyl-benzopyran), HP2 (7-hydroxy-6-isobutyryl-5-methoxy-2,2-dimethyl-benzopyran) and HP3 (5-hydroxy-6-isobutyryl-7-methoxy-2,2-dimethyl-benzopyran) (Ferraz et al., 2001). Subsequently, these benzopyrans were also isolated from H. ternum (Ferraz et al. 2005c). Using the in vitro micropropagation technique, Bernardi et al. (2005a) managed to get the seedlings of H. polyanthemum to produce benzopyrans. These plantlets were successfully acclimatized and showed the same benzopyran profile as in wild plants (Bernardi et al., 2005).

Tannins and flavonoids: The quantification analysis showed tannin levels between 5 and 16%; H. myrianthum (5.1%), H. caprifoliatum (6.4%), H. polyantemum (6.7%), H. carinatum (9.1%), H. connatum (11.5%), H. ternum (16.7%) (Dall 'Agnol et al., 2003). The non tannin phenolic fraction of these species was analyzed for the presence of flavonoid glycosides. Compounds usually cited in the literature were found: hyperoside, quercitrin, and isoquercitrin guaijaverina (Dall 'Agnol et al., 2003).

Benzophenones: From the aerial parts of H. carinatum, we isolated two novel benzophenones, carifenona A and B carifenona (Bernardi et al., 2005).

Essential oils: The species of Hypericum are characteristically aromatic, in the species H. caprifoliatum, H. polyanthemum, H. myrianthum, H. carinatum, H. connatum and H. ternum the amount of volatile oil varies from 0.1% to 0.5%. In these species, sesquiterpenes are in higher concentration than monoterpenes and all have alkanes, mainly nonane and undecane (Ferraz et al., 2005a).

After phytochemical investigation of eight species, H. brasiliense, H. carinatum, H. caprifoliatum, H. connatum, H. cordatum, H. myrianthum, H. polyanthemum and H. piriai, Ferraz et al. (2002b) verified the absence of hypericin in all species. This result is consistent with the chemotaxonomic division proposed by Robson (1990), where the production of hypericin is related to the presence of black glands on the leaves. The Brazilian species, which belong to the sections and Brathys Trigynobrathys have only pale glands, which produce no hypericin.

The species H. caprifoliatum, H. carinatum, H. connatum, H. myrianthum, H. piriai, H. polyanthemum and H. cordatum, and three isolated benzopyrans of H. polyanthemum (HP1, HP2, and HP3) were tested for inhibitory activity of monoamine oxidase A and B in preparations of mitochondria from rat brain. Among the extracts that showed significant inhibition only for MAO_{A}, the ones with higher activity at a concentration of 1.5×10⁻² mg/ml were the chloroform extract of H. caprifoliatum (83%) and H. polyanthemum (82%) and petroleum ether extract of H. piriai (90%), among benzopyrans only HP3 (IC₅₀ MAO_{A}=22.2 μM) showed significant activity (Gnerre et al. 2001). In vitro results were not confirmed in vivo, since the only species that showed a reduction in the immobility time of mice was H. caprifoliatum, but not the chloroform extract, but the petroleum (Daudt et al., 2000; Gnerre et al., 2001).

The possible mechanism of action of H. caprifoliatum is being investigated and, so far, the data indicate the involvement of the dopaminergic system. It was found that the antiimmobility effect of the lipophilic extract (270 mg/kg/day, p.o.) is prevented by pretreatment with sulpiride (50 mg/kg, i.p.) (D2 antagonist), this extract (90 mg/kg, p.o.) enhances the hypothermia caused by apomorphine (16 mg/kg, s.c.) and actions on the reuptake of monoamines, binding to transporters, binding of [³⁵S] GTPγS stimulated by agonists and the HPA axis are equivalent to those of H. polyanthemum.

Other pharmacological activities found for species native of Rio Grande do Sul were antibacterial for H. brasiliense (Rocha et al., 1995, Rocha et al., 1996), H. caprifoliatum, H. myrianthum, H. polyanthemum and H. ternum (Dall'Agnol et al., 2003, 2005), H. ternum presented antifungal activity (Fenner et al., 2005); antiproliferative activity was observed for H. caprifoliatum, H. myrianthum, H. ternum and benzopyrans isolates of H. polyanthemum (Ferraz et al., 2005 b, c); H. connatum showed antiviral activity (Schmitt et al., 2001); carifenona A isolated from H. carinatum demonstrated antioxidant effects (Bernardi et al., 2005); H. caprifoliatum, H. polyanthemum (Viana et al., 2003) and H. brasiliense (Mendes et al., 2002) showed antinociceptive activity.

Hypericum polyanthemum

This is a plant never studied before. Its chemical composition is different from Hypericum perforatum, which is the plant of the genus used by the pharmaceutical industry worldwide. H. perforatum has two substances considered major and which are the basis for the standardization of medicines hypericin and hyperforin (B). The species that we have studied—H. polyanthemum—do not present hypericin and its derivatives phloroglucinol (A) have a quite different chemical structure from hyperforin.

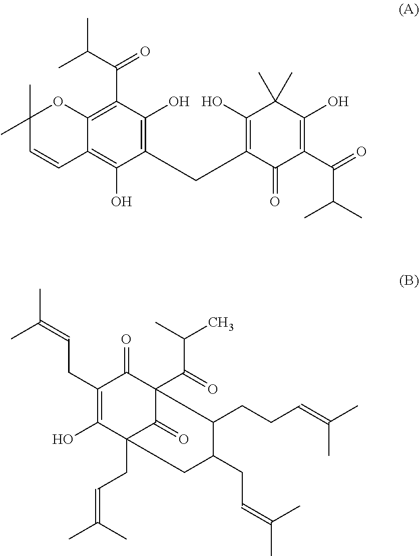

This may represent an important advantage, because the presence of hypericin and hyperforin is related to the occurrence of problems limiting the use of H. perforatum, as photosensitivity and drug interactions, respectively.

The products obtained with H. polyanthemum, especially HP4 (uliginosin B) seem to have more selectivity for inhibition of dopamine reuptake than the extracts of H. perforatum, at least with respect to the biogenic amines serotonin and norepinephrine. The more potent effect on the activation of the dopaminergic system may be of interest for the development of antidepressant drugs more selective for certain subtypes of depression or patients resistant to available therapeutic arsenal, as well as for the treatment of diseases that have depression as a comorbidity (or vice versa), e.g. Parkinson's disease. In addition, repeated treatment with the extract affects stress-related responses, with a different mechanism of action of antidepressants available, which may also be significant in relation to resistant patients.

The main advantage is the possibility of using a native plant of this state (Rio Grande do Sul) to obtain molecules structurally different from those already known, which can be used as drugs, models to obtain drugs or as tools to study the monoaminergic system, also this species may be used by the pharmaceutical industry for the development of herbal drugs with antidepressant activity similar to that of Hypericum perforatum, which is a raw material imported and expensive. The protection of the use of H. polyanthemum lipophilic extracts to obtain products with monoaminergic action can provide a breakthrough in the development of herbal and plant products with Brazilian origin. Similar products on the market are: antidepressant whose mechanism of action involves the inhibition of neuronal reuptake of monoamines and antidepressant drugs made with H. perforatum standardized extracts (in hypericin and hyperforin).

In vitro, uliginosina B (obtained from H. polyanthemum, object of the present invention) and hyperforin (obtained from H. perforatum, technology currently used) inhibit synaptosomal reuptake of serotonin, norepinephrine, and dopamine. However, the effect of hyperforin on the reuptake of neurotransmitters appears to be less specific than that of uliginosina B, which has IC₅₀ for dopamine almost three times smaller than for NA and 5-HT. In addition to the monoamines, hyperforin also inhibits the synatosomal reuptake of GABA and glutamate, supporting the idea of a rather non-selective action.

The occurrence of this molecular pattern is possibly limited (taxonomic marker) to South American species. Moreover, hypericin was not detected in H. polyanthemum (see previous item). Hyperforin and hypericin have been considered responsible for two potentially limitations to the treatment with H. perforatum: a large number of drug interactions (via induction of cytochrome P450) and photosensitization, respectively.

Considering the antidepressants, all substances currently on the market inhibit the uptake of monoamines by binding to their neuronal transporters, while uliginosin inhibits the reuptake without binding to the monoaminergic transporters, which was also observed for hyperforin and ad-hyperforin. So phloroglucinols differ in their mode of action compared to other antidepressants on the market. This feature can be explained by molecular pattern of phloroglucinols derivatives present in the species evaluated and by the structural requirements for binding to monoaminergic receptors and transporters, phloroglucinol derivatives do not have nitrogen atoms that are considered essential for this binding (FIG. 4).

The HPA axis may be an important target for antidepressant action. The hyperactivity of the hypothalamo-pituitary-adrenal (HPA) in depressed patients can be corrected by treatment with antidepressants. In animals, antidepressants reduce cortical and serum corticosterone levels, while the cyclo-hexane extract (POL) reduced only cortical levels, which may represent a different and more selective mechanism.

The particular mode of action of H. polyanthemum and of uliginosin B put them in perspective of a more comprehensive view of antidepressants mechanism of action. There is a consensus among research groups on depression that the increase of monoamines in the synaptic cleft caused by inhibition of its transport does not explain alone the full range of effects of antidepressants. Moreover, many authors consider that continuing the “me too” approach (different molecular substances, but with the same mechanism of action) will not result in truly innovative drugs which may improve some limitations of current treatment, as the delay of at least two weeks to the therapeutic effect while the adverse effects occur from the first dose, and patients resistant to different antidepressants.

The preclinical studies conducted by our group with extracts and/or molecules obtained from species of H. polyanthemum demonstrate clearly the potential use for the development of antidepressants. This activity is supported by other findings, already found and published by our group to the species H. caprifoliatum, also native to RS, as well as the antidepressant activity of H. perforatum, European species. The present invention relates to the use of lipophilic extracts of H. polyanthemum for the production of antidepressant phytomedicines. Uliginosina B can be used in the production of antidepressants or drugs with dopaminergic activity. This substance can also be used as a prototype molecule for the synthesis of drugs with activity on the monoaminergic system and on the hypothalamic-pituitary-adrenal axis.

A patent literature search revealed some relevant documents which will be described below.

The document U.S. Pat. No. 6,346,282 discloses a pharmaceutical composition for targeted treatment of nervous system disorders such as anxiety disorders, irritability or depression comprising the synergistic use of a Hypericum extract with an enhancer as acetyl-L-carnitine.

The document U.S. Pat. No. 6,472,439 reports a pharmaceutical composition comprising a plant extract and two carriers. Specifically, the plant extract is an Hypericum spp extract, one of the carriers is chosen from the group consisting of polyethylene glycol, polyvinyl alcohol, polyvinyl pirolidone among other compounds and the second carrier is a compound insoluble in alcohol. Specifically, the non-volatile part of this pharmaceutical composition is bound in a microdispersa form.

The document U.S. 2003/0012824 reports a pharmaceutical composition comprising an anti-anxiety agent, an anti-acid compound and an inducer of mental alertness. Specifically, the anti-anxiety agent is a plant extract, among them the extract of Hypericum perforatum, the anti-acid agent is selected from the group comprising aluminum carbonate, aluminum hydroxide, among others, and a promoter of mental alertness is chosen from the group comprising plant extracts that promote blood flow.

The document U.S. 2006/0167074 describes a pharmaceutical composition for the prevention, treatment or inhibition of psychiatric disorders, particularly schizophrenia and depressive disorders. Specifically, the composition described herein comprises a combination of an antidepressant or neuroleptic agent with an inhibitor of COX-2 or a pro-drug thereof.

The document U.S. 2007/0231405 reports a pharmaceutical composition comprising one vitamin, one mineral and one plant species. Specifically, the herb used is Hypericum perforatum.

The document WO 1999/64388 reports a pharmaceutical composition for treating depressive disorders. Specifically, the pharmaceutical composition of this document includes the use of hyperforin derivatives.

The document U.S. 2007/0190187 reports a pharmaceutical composition for the treatment of psychiatric disorders that includes the use of various plant extracts, among them the extract of Hypericum polyanthemum.

None of the documents cited above disclose a plant extract of Hypericum polyanthemum comprising compounds belonging to the class of phloroglucinols and/or benzopyrans.

Therefore, it was not found in the literature any document which anticipates or even suggests the specificities of the present invention.

### SUMMARY OF THE INVENTION

One object of the present invention is a plant extract of Hypericum polyanthemum containing compounds belonging to the class of phloroglucinols and/or benzopyrans.

In a preferred embodiment the phloroglucinol of the invention is uliginosin B and/or its derivatives.

The plant parts of H. polyanthemum can be chosen in the group consisting of leaves, stem, root, fruit, seeds, flowers and a mixture thereof. Specifically, the present invention uses the aerial parts of H. polyanthemum.

Yet another object of the present invention is a pharmaceutical composition comprising:

a) at least one plant extract of Hypericum polyanthemum comprising compounds belonging to the class of phloroglucinols and/or benzopyrans, and

b) a pharmaceutically acceptable vehicle.

Specifically, the pharmaceutical composition of the present invention can comprise a plant extract containing uliginosin B and/or its derivatives and is intended for the treatment of nervous system disorders, specifically the treatment of depression.

Another object of the present invention is a process for producing an extract of Hypericum polyanthemum comprising compounds belonging to the class of phloroglucinols and/or benzopyrans comprising the steps of maceration of at least a portion of the plant H. polyanthemum in an organic solvent, typically cyclohexane, where the ratio plant mass (g):organic solvent volume (mL) is comprised between 1:1 and 1:50.

### BRIEF DESCRIPTION OF FIGURES

FIG. 1 shows the effect of H. polyanthemum cyclohexane extract POL) at doses of 180 and 270 mg/kg and imipramine (IMI) 20 mg/kg, p.o. on the immobility time of mice in the forced swimming test and antagonism of POL270 effect with SCH 23390 (SCH) 15 μg/kg and sulpiride (SUL) 50 mg/kg. Results are expressed as mean±SEM. * Significant difference compared to the SAL group (water+polysorbate 80, 5%, 1 ml/kg, p.o.) # significant difference compared to POL270 (ANOVA F_{(7,79)}=5.9, p<0.001).

FIG. 2 shows the effect of uliginosina B (HP4, 90 mg/kg, p.o.) and imipramine (IMI, 20 mg/kg, p.o.) on immobility time of mice in the forced swimming test. Results are expressed as mean±SEM. *Significant difference compared to the SAL group (water+polysorbate 80, 5%, 1 ml/kg, p.o.) (ANOVA, F_{(2,29)}=15.5, p<0.001).

FIG. 3 shows the effect of three days of treatment with imipramine (IMI 20 mg/kg), bupropion (BUP 30 mg/kg), H. polyanthemum (POL 360 mg/kg) or 5% polysorbate in water (SAL) on plasma (A) and cortical (B) corticosterone levels in mice subjected or not to forced swimming. Data are presented as mean±SEM. *p<0.01 compared to respective group without swimming; #p<0.01 compared to SAL with swimming. Legend: (A) X axis—corticosterone levels (μg/100 mL plasma); Blank values: no swimming; values hatch: with swim, (B) X axis—cortical Corticosterone (pg/100 mg tissue).

### DETAILED DESCRIPTION OF THE INVENTION

The examples described below are not intended to limit the scope of this invention, but only to show one way of working it.

Plant Material

The plant material used in this invention is any plant material from plants of the genus Hypericum, especially Hypericum polyanthemum, such as root, stem, leaf, flower, fruit, seed, and mixtures thereof.

Typically, the present invention uses the aerial parts of H. polyanthemum.

Compounds Belonging to the Class of Phloroglucinols and/or Benzopyrans

The compounds belonging to the class of phloroglucinols include, but are not limited to, hiperbrasilol-A, hiperbrasilol-B, isohiperbrasilol-B, hiperbrasilol-C, isouliginosina B, japonicina, uliginosin A and uliginosin B, as well as their salts, solvates and/or carbohydrates. The compounds belonging to the class of benzopyrans include, but are not limited to benzopyrans HP1, HP2, HP3 and their salts, solvates and/or carbohydrates.

The preferred compound of this invention is the uliginosin B, which has the structural formula below.

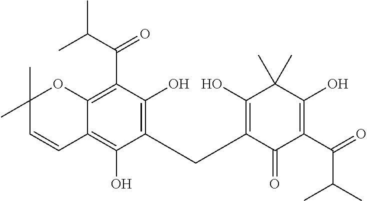

Extraction Process

The extraction process of this invention is accomplished by any extraction process as described in the prior art, such as the use of Soxhlet apparatus, maceration, among others. The solvent used is a lipophilic solvent, for example, cyclohexane.

Typically, the present invention uses Soxhlet extraction and manual extraction with a separating funnel.

In particular the leaves of H. polyanthemum are macerated for 24 hours 3 times, and then filtered.

Purification Process

The purification of the compounds present in each extract is carried out by any purification method already used in the prior art such as thin layer chromatography and column chromatography, among other possibilities.

Typically, the purification process used in this invention is column chromatography and thin layer chromatography.

Pharmaceutical Composition

For the purposes of this invention, “pharmaceutical composition” refers to any composition containing an active ingredient, with prophylactic, palliative and/or healing effect, acting in order to maintain or restore homeostasis, and can be administered in a topical, parenteral, enteral and/or intrathecal form.

The term “pharmaceutically acceptable” is used here to refer to compounds, materials, compositions, and/or dosage forms that are within the scope of medicine, suitable for use in contact with the tissues of humans and animals without excessive toxicity irritation, allergic response, or other problem or complication, commensurate with a reasonable relationship of benefit/risk.

The composition of the present invention can be administered in oral dosage form such as tablets, capsules (each of which includes sustained release formulations or time-release) tablets, powders, granules, elixirs, tinctures, suspensions, syrups, and emulsions. They can be administered alone but will generally be administered with a pharmaceutical vehicle selected on the basis of route of administration and standard pharmaceutical practice.

The pharmaceutical composition of the present invention comprises:

a) at least one plant extract of Hypericum polyanthemum comprising compounds belonging to the class of phloroglucinols and/or benzopyrans, and

b) a pharmaceutically acceptable vehicle.

Typically, the pharmaceutical composition of this invention comprises a plant extract containing uliginosin B and/or its derivatives and is intended for the treatment of nervous system disorders, specifically the treatment of depression.

The present invention also relates to the use of an extract of Hypericum polyanthemum having at least one compound belonging to the class of phloroglucinols and/or benzopyrans for the manufacture of a drug used to treat mood disorders and/or depression.

The present invention also relates to an extract of Hypericum polyanthemum having at least one compound belonging to the class of phloroglucinols and/or benzopyrans for use in a method of treatment of mood disorders and/or depression.

The present invention also relates to a method for treating mood disorders and/or depression comprising the step of administering to a patient an effective amount of a extract of Hypericum polyanthemum having at least one compound belonging to the class of phloroglucinols and/or benzopyrans.

### Example 1

### Preparation of Plant Extract

### Example 1.1

### Plant Material and Chemical Study

The aerial parts of H. polyanthemum were collected in Caçapava do Sul/RS. The specimens of the plant material were prepared for identification and recorded in the ICN herbarium (Herbarium of the Botany Department—Instituto de Biociéncias—UFRGS) under number Bordignon et al. 1429 (H. polyanthemum). The material, immediately after collection, was selected, dried in an airy atmosphere, protected from direct light, and torn up manually.

### Example 1.2

### Obtaining Cyclohexane Extract (POL)

The aerial parts, dried at room temperature and in the dark and torn up were subjected to a soaking operation (3×24 h) with cyclohexane at a ratio of 1 g of plant material per 10 mL of solvent. After each extraction, the mixture was filtered and the cake underwent the same operation twice.

### Example 2

### Isolation and Identification of Chemical Constituents

The main components of POL were obtained by column chromatography using gradients of hexane/ethyl acetate, followed by thin layer chromatography on preparative silica gel GF254 using chloroform/hexane (3.5:1 V/V) as eluent. One of the major components was analyzed by magnetic resonance ¹H, ¹³C (CDCl₃, 400 MHz) and characterized as uliginosin B (HP4), a derivative of phloroglucinol and filicinic acid. The main components of POL and CLOR (chloroform extract) were obtained by thin layer chromatography on preparative silica gel GF254 using chloroform as eluent and analyzed by magnetic resonance ¹H, ¹³C (CDCl₃, 400 MHz). We identified three benzopyrans: HP1 (6-isobutyryl-5,7-dimethoxy-2,2-dimethyl-benzopyran), HP2 (7-hydroxy-6-isobutyryl-5-methoxy-2,2-dimethyl-benzopyran) and HP3 (5-hydroxy-6-isobutyryl-7-methoxy-2,2-dimethyl-benzopyran).

### Example 3

### Pharmacological Study of the Antidepressant Activity

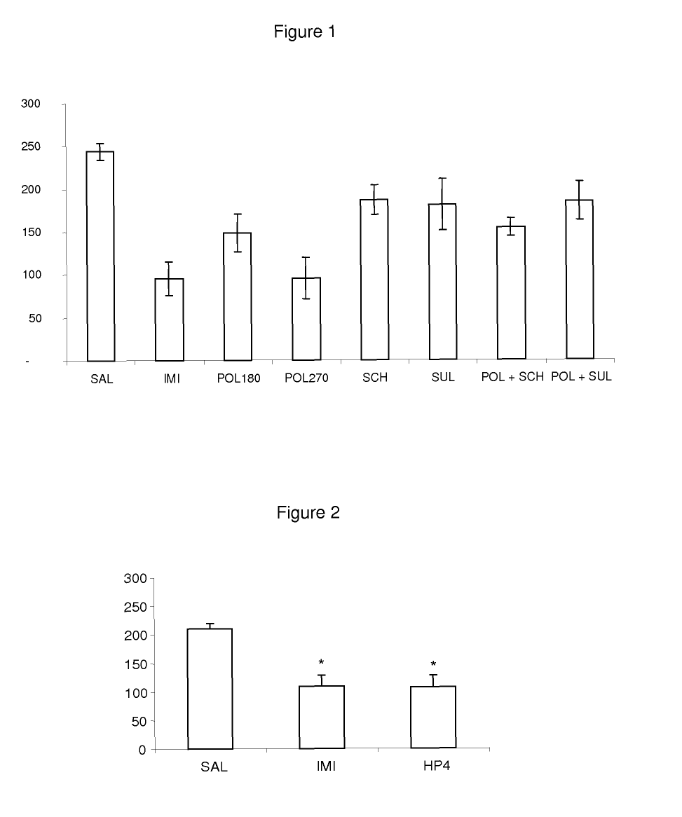

POL and its phloroglucinol derivative uliginosin B (HP4) showed activity in an animal model of depression—Porsolt's forced swimming test (FST)—in rats and CF1 mice. POL was active in rats (270 mg/kg/day, p.o.) and mice (180, 270 and 360 mg/kg, p.o.). The action of POL in the FST was blocked by administration of SCH 23390 (dopamine D1 antagonist) 15 mg/kg, i.p., and sulpiride (D2 dopamine antagonist) 50 mg/kg, ip, (FIG. 1). HP4 was active at a dose of 90 mg/kg, p.o. in mice (FIG. 2).

### Example 3.1

### Effect on Monoamine Transporters

The effect of HP4 (3×10⁻⁴-3×10⁻¹¹ g/mL) on synaptosomal reuptake of dopamine ([³H]-DA), noradrenaline ([³H]-NA) and serotonin ([³H]-5HT) in synaptosomes prepared from striatum, hypothalamus and frontal cortex, respectively. HP4 inhibited the synaptosomal uptake of DA more potently (IC₅₀ 90±38 nM) than 5-HT (IC₅₀ 252±13 nM) and NA (IC₅₀ 280±48). These results suggest that the potential antidepressant effect of H. polyanthemum is related to the dopaminergic system. However, this effect does not appear to be dependent on a direct action of the substances on the monoamine transporter, since different concentrations HP4 (3×10⁻⁷-3×10⁻¹¹ g/mL) did not affect the binding of [³H]-mazindol, [³H]-nisoxetina and [³H]-citalopram to the sites of uptake of DA, NA and 5-HT, respectively, in membranes prepared with the same structures mentioned above. This profile differs from other antidepressants, which inhibit the reuptake of monoamines through competition for binding site on the transporter.

### Example 3.2

### Effect of POL and HP4 on Activation of G Proteins

To evaluate the effect of POL on DA, NA, 5-HT and opioids receptors, we used the technique of binding of [³⁵S] GTPγS stimulated by DA, NA, 5-HT and DAMGO in membranes prepared from striatum, hypothalamus, frontal cortex and thalamus of rats, respectively. Acute treatment of mice with POL (90 mg/kg and 270 mg/kg, p.o.) significantly increased the binding of [³⁵S] GTPγS stimulated by DA, NA and 5-HT, while 5 days of treatment with 90 mg/kg, decreases this binding. None of the regimens affected the binding stimulated by DAMGO, an opioid agonist, which shows a selective effect for monoaminergic receptors. This time profile of action coincides with the modern theories that the antidepressant effect is related to neuroadaptation processes.

Direct incubation of membranes with HP4 did not alter the binding of [³⁵S] GTPγS to its site. These results demonstrate that treatment with POL causes changes in monoamine transmission, but these are not due to direct effects of HP4 on the receptors. These changes can occur through other regulatory mechanisms or are related to HP4 metabolites produced after administration in vivo of POL. Table 1 shows the values for power (EC₅₀) and maximal effect (Eₘₐₓ) of binding of [³⁵S] GTPγS stimulated by agonists after different treatment regimens with saline and POL.

TABLE 1
Effect of different treatment regimens with POL on the potency
(EC₅₀) and maximum effect (Eₘₐₓ) of [³⁵S] GTPγS binding stimulated by agonists.

| | | DA | NA | 5-HT | DAMGO |
|---|---|---|---|---|---|
| | | EC₅₀ (μM) | | | |
| Salina | T1 | 25 ± 6.2 | 42.5 ± 3.6 | 156 ± 25 | 0.24 ± 0.04 |
| | T2 | 18.1 ± 2.4 | 39.4 ± 6.5 | 135 ± 26 | 0.37 ± 0.07 |
| | T3 | 5 ± 1 | 10.4 ± 2.6 | 63.2 ± 25.9 | 1.6 ± 0.3 |
| POL | T1 | 7.6 ± 0.8** | 7.2 ± 1.3** | 13.4 ± 2.6** | 0.35 ± 0.08 |
| | T2 | 0.32 ± 0.05*** c | 0.28 ± 0.11*** b | 0.13 ± 0.03*** c | 1.46 ± 0.47 |
| | T3 | 307 ± 92*** | 189 ± 40** | 534 ± 225*** | 2.1 ± 0.6 |
| | | Emax | | | |
| Saline | T1 | 60.6 ± 2.2 | 48.9 ± 3.9 | 45.3 ± 3.3 | 79.7 ± 4.5 |
| | T2 | 59.3 ± 3.9 | 47.9 ± 5.9 | 33.8 ± 2.7 | 71.6 ± 5.2 |
| | T3 | 83.6 ± 3.7 c | 68 ± 2.3 | 60.3 ± 8.3 | 70.6 ± 5 |
| POL | T1 | 73.2 ± 2.6* c | 66.9 ± 1.5 | 57.1 ± 1.1 a | 77.4 ± 1.3 |
| | T2 | 93.2 ± 3.6*** c | 113.5 ± 17.1*** c | 93.1 ± 10.9*** c | 66.6 ± 3.9 |
| | T3 | 44.8 ± 5.9*** | 40.2 ± 1.1* | 36.1 ± 4.5* | 64.1 ± 1.8 |

T1 - acute treatment (90 mg/kg, p.o.)
T2 - three treatments in 24 h (270 mg/kg, p.o.)
T3 - five days of treatment with 90 mg/kg, p.o.
Mean ± SEM of 4 experiments in duplicate.
*p < 0.05, **p < 0.01, ***p < 0.001, comparisons on the same regimen, significant difference compared to SAL.
a p < 0.05, b p < 0.01, c p < 0.001, comparisons within the same treatment group, significant difference compared with other treatment regimens.

### Example 3.3

### Effect on Serum and Cortical Corticosterone

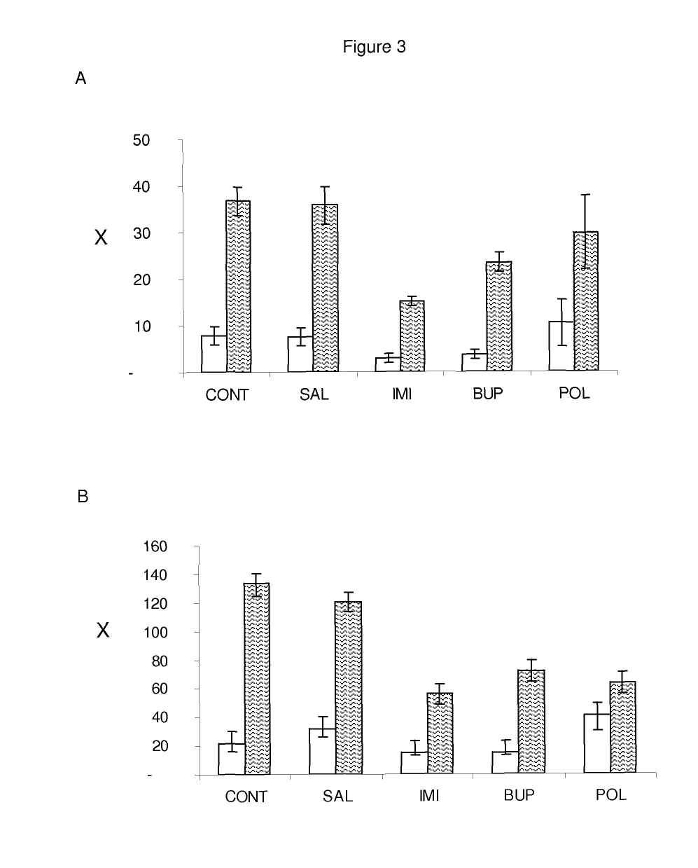

We evaluated the action of POL (360 mg/kg, p.o.) on serum and cortical levels of corticosterone in mice subjected or not to stress caused by forced swimming. The concentration of corticosterone present in the samples was measured by radioimmunoassay. The results showed that three days of treatment with POL decreases significantly the level of cortical corticosterone raised by forced swimming, without affecting the serum levels (FIG. 3), while treatment with antidepressants imipramine and bupropion alters both serum and cortical levels. These results show that repeated treatment with the extract affects stress-related responses, with a different mechanism of action than current drugs.

1. Plant extract characterized by being obtained from plants belonging to the species Hypericum polyanthemum and comprising at least one compound belonging to the class of phloroglucinols and/or benzopyrans.

2. Plant extract, according to claim 1, characterized by being obtained from plant parts selected from the group consisting of root, stem, leaf, flower, fruit, seed, and combinations thereof.

3. Plant extract, according to claim 1, characterized in that the compound belonging to the class of phloroglucinols is chosen from the group consisting of iso-uliginosin B, uliginosin A, uliginosin B, salts and solvates and/or hydrates, and mixtures thereof.

4. Plant extract, according to claim 3, characterized in that the compound belonging to the class of phloroglucinols is uliginosin B.

5. Plant extract, according to claim 1, characterized in that the compound belonging to the class of benzopyrans is chosen from the group consisting of the benzopyrans HP1, HP2, HP3, and mixtures thereof.

6. Plant extract, according to claim 1, characterized in that it is obtained from the aerial parts of H. polyanthemum.

7. Production process of a plant extract comprising the steps of:
a. extracting at least one phloroglucinol and/or benzopyran from a plant belonging to the species Hypericum polyanthemum in an organic solvent; and
b. purifying the extract obtained.

8. Process, according to claim 7, characterized in that the ratio of plant mass (g):organic solvent volume (mL) is between 1:1 and 1:50.

9. Process according to claim 8, characterized in that the ratio is 1:10.

10. Process according to claim 7, characterized by being obtained from plant parts selected from the group consisting of root, stem, leaf, flower, fruit, seed, and combinations thereof.

11. Process according to claim 7, characterized in that said at least one phloroglucinol is chosen from the group consisting of iso-uliginosin B, uliginosin A, uliginosin B, salts and solvates or hydrates, and mixtures thereof.

12. Process according to claim 11, characterized in that said phloroglucinol is uliginosin B.

13. Process according to claim 7, characterized in that said at least one benzopyran is chosen from the group consisting of the benzopyrans HP1, HP2, HP3, and mixtures thereof.

14. Process according to claim 7, characterized in that the aerial parts of H. polyanthemum are used.

15. Process according to claim 7, characterized in that the organic solvent is cyclohexane.

16. Process according to claim 7, characterized in that the purification step comprises the submission of the extract obtained in a) to a chromatography.

17. Process according to claim 16, characterized in that said chromatography is thin-layer chromatography and/or column chromatography.

18. Pharmaceutical composition characterized in that it comprises:
a. an extract of Hypericum polyanthemum with at least one compound belonging to the class of phloroglucinols and/or benzopyrans, and
b. a pharmaceutically acceptable vehicle.

19. Composition according to claim 18, characterized in that said extract is obtained from plant parts selected from the group consisting of root, stem, leaf, flower, fruit, seed, and combinations thereof.

20. Composition according to claim 18, characterized in that the compound belonging to the class of phloroglucinols is chosen from the group consisting of iso-uliginosin B, uliginosin A, uliginosin B, salts and solvates or hydrates, and mixtures thereof.

21. Composition according to claim 20, characterized in that the compound is uliginosin B.

22. Composition according to claim 18, characterized in that the benzopyran is chosen from the group consisting of the benzopyrans HP1, HP2, HP3, and mixtures thereof.

23. Composition according to claim 18, characterized in that said extract is obtained from H. polyanthemum aerial parts.

24. (canceled)

25. (canceled)

26. A method for treating mood disorders and/or depression comprising the step of administering to a patient an effective amount of a extract of Hypericum polyanthemum having at least one compound belonging to the class of phloroglucinols and/or benzopyrans.
